(19) United States
(12) Patent Application Publication
(10) Pub. No.: US 2026/0185139 A1
(43) Pub. Date: Jul. 2, 2026

(54) USEFUL COMBINATIONS OF RESTRICTION ENZYMES

(71) Applicant: NUCLEIX LTD., Rehovot (IL)

(72) Inventors: Danny FRUMKIN, Rehovot (IL); Revital KNIRSH, Rosh HaAyin (IL)

(73) Assignee: NUCLEIX LTD., Rehovot (IL)

(21) Appl. No.: 18/867,774

(22) PCT Filed: May 22, 2023
(86) PCT No.: PCT/IL2023/050518

§ 371 Date: Nov. 20, 2024

(30) Foreign Application Priority Data
May 22, 2022 (IL) 293202

(51) Int. Cl. C12Q 1/6806; C12Q 1/34; C12Q 1/48; C12Q 1/686; C12Q 1/6869

(52) U.S. Cl. CPC C12Q 1/6806; C12Q 1/34; C12Q 1/48; C12Q 1/686; C12Q 1/6869; C12Y 207/07; C12Y 301/21004; C12Q 2600/112; C12Q 2600/154

(57) ABSTRACT

Various compositions and methods are disclosed in which a plurality of restriction enzymes, including methylation-sensitive and/or methylation-dependent restriction enzymes, are used for the analysis of cfDNA. Useful combinations can be inactivated by heating to 65° C., ideally for longer than 15 minutes. Restriction digestion of cfDNA can occur for 11 hours or less. Digestion may be followed by amplification and/or sequencing steps.

All documents and online information cited herein are incorporated by reference in their entirety.

### TECHNICAL FIELD

The invention is in the field of analysing methylation of cytosine residues in DNA, and is particularly useful for analysing human cell-free DNA e.g. as found in plasma.

### BACKGROUND

Various techniques are known for analysing methylation of cytosine residues in DNA. One common method involves bisulfite conversion, in which unmethylated cytosines are converted to uracil using bisulfite. The converted DNA is then analysed, and a comparison of bisulfite-treated and bisulfite-untreated DNA reveals which cytosine residues were not converted to uracil (and thus were methylated). One major drawback with this technique is that bisulfite conversion is chemically harsh, leading to high levels of degradation of source material, which is a problem when using small quantities of source DNA. The chemical conversion is also biased, and inherently noisy.

Another technique uses a methylation-sensitive restriction enzyme (MSRE) whose activity is blocked if a cytosine within a CpG site in the enzyme's recognition sequence is methylated. Various MSRE-based techniques are available, using either single enzymes or combinations. For instance, the HELP assay uses a combination of HpaII and MspI. The recognition sequence for both of these enzymes is CCGG, but HpaII is methylation-sensitive. A comparison of the digestion products for the two enzymes can thus reveal which CCGG sites were CpG-methylated. Other MSRE-based assays using multiple enzymes are known, including methods using three or four (or more) different enzymes.

It is also possible to use a methylation-dependent restriction enzyme (MDRE) which digests its recognition sequence only if a cytosine is methylated i.e. the inverse of a MSRE-based assay.

These enzyme-based techniques have also been used to analyse methylation of cell-free DNA (cfDNA), as in the EpiCheck platform marketed by Nucleix. The use of multiple enzymes for digestion of cfDNA has also been reported. For instance, mixtures of HhaI, HpaII and exonuclease I have been used to digest cfDNA, and mixtures of two or three of BstUI, HhaI, and/or HpaII have been used for analysing fetal cfDNA in maternal blood. Methods for digesting cfDNA using BstUI alone or in combination with HhaI, HpaII, or HpaII+HinP1I are known, as are methods using Bsh12361+HhaI, Bsh12361+HpaII+HinP1I, HinP1I+HhaI, HhaI+HpaII, or HhaI+AccII, as well as methods using HpaII+AciI+HpyCH4IV and HinP1I. Also known are DNA digestion methods using BstUI+HpaII, HpaII+CfoI or mixtures of two or three of AccII, HpaII and HpyCH4IV, as are methods using BstUI+MluI, BstUI+HpaII or NauI+MbuBI. Various of these methods involve a downstream PCR step, so it is necessary to inactivate the MSRE(s) prior to PCR so that the amplicons (which will be unmethylated) are not digested.

There remains a need for further MSRE and/or MDRE combinations which are useful for digesting cfDNA and which can offer various advantages over combinations which have already been used, including in techniques which include downstream PCR. There is also a need for further techniques for analysing CpG methylation using restriction enzyme combinations and which can offer advantages over known methods.

### SUMMARY

The invention provides a method for digesting cfDNA using a combination of restriction enzymes comprising HinP1I and AciI, wherein the method comprises steps of: (i) digesting the cfDNA with the restriction enzymes; and (ii) inactivating the restriction enzymes by heating for longer than 15 minutes. Inactivating the restriction enzymes by heating for 20 minutes or longer is preferred. This period of inactivation can achieve complete inactivation, to ensure that residual enzymatic activity does not persist into downstream steps, unlike the 15 minutes of heat inactivation used in some known methods.

The invention also provides a method for digesting cfDNA using a combination of restriction enzymes comprising HinP1I and AciI, wherein the method comprises steps of: (i) digesting the cfDNA with the restriction enzymes for 11 hours or less; and (ii) completely inactivating the restriction enzymes by heating. 11 hours of digestion is more than adequate for digestion of all cfDNA in a typical sample, and the 14 hour or 16 hour digestion times in some known methods are unnecessarily long.

Although 11 hours or less of digestion is adequate for complete digestion of cfDNA in a typical sample obtained from blood collected in a typical collection tube containing an anticoagulant, such as a K₂EDTA collection tube, digestion has been found to be inhibited in some types of collection tube. In particular, this inhibition can be seen in collection tubes which contain an anticoagulant and an agent to inhibit genomic DNA from white blood cells being released into the plasma component of the blood sample, such as a BCT from Streck (see below). When blood has been stored in such tubes it may thus be useful to increase digestion times compared to blood which was stored in a K₂EDTA collection tube. Thus the invention also provides a method for digesting cfDNA using a combination of restriction enzymes comprising HinP1I and AciI, wherein the method comprises steps of: (i) providing a blood sample contained within a collection tube that includes an anticoagulant and an agent to inhibit genomic DNA from white blood cells in the sample being released into the plasma component of the blood sample; (ii) preparing plasma from the blood sample; and (iii) digesting the cfDNA with the restriction enzymes for at least 2 hours. This method can further comprise a step of (iv) inactivating the restriction enzymes by heating, as disclosed elsewhere herein. The use of this type of collection tube advantageously enables blood samples to be stored and/or shipped (e.g. at room temperature) after being taken, while inhibiting contamination of cfDNA by genomic DNA which can otherwise be released into plasma from white blood cells during storage. The one hour digestion time used in some known methods after blood collection in such a tube from pregnant mothers has sometimes been found to be too short for achieving consistent and reliable results with cfDNA, and step (iii) can involve digestion for longer than 2 hours (e.g. for at least 4, 6, 8, 10, 12, 14 hours or more e.g. for about 16 hours) and ideally long enough to provide substantially complete digestion of the cfDNA. Contrary to previous reports that certain cfDNA blood collection tubes are unsuitable for downstream analysis of methylated sequences in cfDNA, it has now been found that blood samples stored in these tubes can indeed be subjected to cfDNA analysis as disclosed herein, particularly when digestion occurs for 2 hours or more.

The invention similarly provides a method for digesting cfDNA using a combination of restriction enzymes comprising HinP1I and AciI, wherein the method comprises a step of digesting the cfDNA with the restriction enzymes for at least 2 hours, wherein the cfDNA is derived from plasma prepared from a blood sample that was contained within a collection tube that includes an anticoagulant and an agent to inhibit genomic DNA from white blood cells in the sample being released into the plasma component of the blood sample. This method can further comprise a step of inactivating the restriction enzymes by heating, as disclosed elsewhere herein.

The invention similarly provides a method for digesting cfDNA using a combination of restriction enzymes comprising HinP1I and AciI, wherein the method comprises steps of: (i) preparing plasma from a blood sample which was contained within a collection tube that includes an anticoagulant and an agent to inhibit genomic DNA from white blood cells in the sample being released into the plasma component of the blood sample; and (ii) digesting the cfDNA with the restriction enzymes for at least 2 hours. This method may comprise either or both of: before (i), a step of receiving the collection tube; and/or after step (ii), inactivating the restriction enzymes by heating, as disclosed elsewhere herein.

The invention also provides a method for analysing cfDNA, wherein the method comprises steps of: (i) digesting the cfDNA with using a combination of restriction enzymes comprising HinP1I and AciI; and (ii) sequencing of the digested cfDNA. In particular, step (ii) can involve next generation sequencing, in which digested cfDNA is converted into a sequencing library, and sequencing reactions are then performed on the library.

The invention similarly provides a method for analysing cfDNA, wherein the method comprises a step of sequencing a digested cfDNA sample, wherein the sample has previously been digested using a combination of restriction enzymes comprising HinP1I and AciI.

The invention also provides a method for analysing cfDNA, wherein the method comprises steps of: (i) digesting the cfDNA for 11 hours or less using a combination of restriction enzymes comprising HinP1I and AciI; (ii) completely inactivating of the restriction enzymes; and (iii) performing real-time PCR on the digested cfDNA. As noted above, 11 hours of digestion is adequate for digestion of typical amounts of cfDNA, particularly when followed by downstream real-time PCR analysis.

The invention similarly provides a method for analysing cfDNA, wherein the method comprises steps of: (i) digesting the cfDNA for 11 hours or less using a combination of restriction enzymes comprising HinP1I and AciI; (ii) completely inactivating the restriction enzymes; and (iii) performing real-time PCR on the digested cfDNA. As noted above, 11 hours of digestion is adequate for digestion of typical amounts of cfDNA, particularly when followed by downstream real-time PCR analysis.

The invention also provides a method for analysing data derived from digested cfDNA, wherein the data comprise real-time PCR quantification cycle data or sequence read data from high-throughput sequencing, and the cfDNA was digested by a method for digesting cfDNA as disclosed herein. The method can be used to provide the methylation status of one or more CpG sites of interest in the cfDNA.

The invention also provides a composition comprising a plurality of restriction enzymes, wherein the plurality consists of MSRE and/or MDRE, and wherein (i) at least two different restriction enzymes in the plurality have different recognition sequences, and (ii) the restriction enzymes can be completely inactivated by heating to 65° C. By recognising different sequences, the number of genomic CpG sites which can be analysed is increased compared to using combinations of enzymes which have the same recognition sequence (e.g. HhaI and HinP1I, as used by Zhao et al., which both recognise GCGC but with different cleavage sites therein). Inactivation at 65° C. is gentler (e.g. in terms of generating undesirable ssDNA) and easier (e.g. less energy intense) than inactivation of mixtures including enzymes such as HpaII, Aval, HaeII, or MluI (which require heating to 80° C. for inactivation, according to suppliers of such enzymes), and provides clear advantages over mixtures which include enzymes that cannot be readily heat-inactivated (as reported for BstUI, PvuI, and HhaI, for example).

This composition may be based on MSREs, without needing MDREs, and so the invention also provides a composition comprising a plurality of MSREs wherein (i) at least two different MSREs in the plurality have different recognition sequences, and (ii) the plurality of MSREs can be completely inactivated by heating to 65° C. This composition may be free from MDRE.

The invention also provides a composition comprising HinP1I and AciI as the only two restriction enzymes in the composition. This pairing of enzymes covers over 99% of CpG islands in the human genome, while being simpler to prepare, with greater precision, than more complex mixtures which have sometimes been used. A composition comprising only two restriction enzymes is advantageous in that it is easier to tailor digestion conditions (e.g., incubation temperature, reaction buffer etc.) to a combination of two enzymes compared to a combination of three or more enzymes without compromising enzyme activity. For example, Bsh1236I, HpaII and HinP1I, as used by Ellinger et al. require different optimal buffers for 100% activity. Whereas ThermoFisher recommends the “Tango buffer” as optimal for its HpaII and HinP1I, for Bsh1236I it recommends “Buffer R”. Preparing a reaction mixture comprising this combination of enzymes using Tango buffer only, as reported by Ellinger et al., thus compromises Bsh1236I activity.

Both HinP1I and AciI show 100% activity at 37° C. and exhibit 100% activity in the same reaction buffer, namely rCutSmart™ (NEB). Both of these enzymes also use the same diluent (diluent A; NEB) and can also be completely inactivated by heating to 65° C.

A composition comprising only two restriction enzymes is also advantageous in downstream library preparation methods that involve the depletion of small DNA fragments, which are not subsequently sequenced. Prior to sequencing small DNA fragments are typically depleted to remove free (i.e., un-ligated) sequencing adapters and/or the adapter dimers which can otherwise interfere with the efficiency and/or quality of DNA sequencing. Where a starting cfDNA molecule is cleaved at more than one site, a greater number of DNA fragments is provided, some of which are very small and are removed (and thus not sequenced) during this step. Increasing the number of restriction enzymes used for digestion increases the likelihood of generating these small DNA fragments, leading to library preparation bias and an underestimation of unmethylated CpG sites. A composition comprising only two restriction enzymes limits this bias.

The invention also provides a composition comprising HinP1I and AciI, wherein the ratio of HinP1I to AciI is at least 1.2:1 (measured in terms of enzymatic units). Using an excess of HinP1I has been found to give better results than a 0.5:1 or 1:1 ratio which has previously been used. Without wishing to be bound by theory, it is believed that an improvement can arise because AciI can cut the human genome more frequently than HinP1I and, as a single cut is enough to impair PCR amplification, less AciI activity is required to achieve the same impairment.

In these various methods and compositions, it is preferred that: (a) the ratio of HinP1I to AciI is at least 2:1; (b) the restriction enzymes are provided with a source of Mg⁺⁺ ions; (c) the restriction enzymes are used at a pH above 7 e.g. in the range of 7.5-8.5; (d) the cfDNA is human cfDNA e.g. human plasma cfDNA; and/or (e) the amount of cfDNA subjected to digestion is between 10-400 ng e.g. between 10-250 ng or between 10-200 ng.

The invention also provides further methods and compositions which include or use these compositions and/or methods, as detailed below.

### DETAILED DESCRIPTION

### Methylation

The methods and compositions disclosed herein are useful for the analysis of DNA methylation, and in particular for analysing the presence or absence of 5-methyl modifications of cytosine in the context of a CG dinucleotide sequence (commonly denoted as ‘CpG’ dinucleotides or ‘CpG sites’) in eukaryotic DNA. CpG sites are not randomly distributed throughout eukaryotic genomes, and are frequently found in clusters known as ‘CpG islands’. These islands have been formally defined (Gardiner-Garden & Frommer (1987) J Mol Biol 196:261-82) as regions which are at least 200 bp long, having 50% or more GC content, and where the observed-to-expected CpG ratio is greater than 60% (i.e. where the number of CpG sites multiplied by the length of the sequence, divided by the number of C multiplied by the number of G, is greater than 0.6). CpG islands are often found near the start of a gene in mammalian genomes, and about 70% of promoters near transcription start sites in the human genome contain a CpG island. Methylation of multiple CpG sites within a promoter's CpG island is generally associated with stable silencing of gene expression from that promoter.

The human genome sequence contains around 28 million CpG sites (per haploid genome), with around 30,000 CpG islands. In any particular nucleated cell some CpG sites will be methylated and others will not. Patterns of methylation can differ between different cells and tissues within a subject, such that a specific CpG can be methylated in one cell or tissue but unmethylated in a different cell or tissue within the same subject.

It is known that tumors can display different methylation patterns compared to non-tumor cells (or compared to other types of tumor). Some sites can become hypermethylated in tumors, while others can become hypomethylated, and the difference in these patterns has been used to aid tumor diagnosis.

### Cell-Free DNA

The methods and compositions disclosed herein are particularly useful for analysing cell-free DNA (cfDNA) i.e. fragmented genomic DNA which is found in vivo in an animal within a bodily fluid rather than within an intact cell. The origin of cfDNA is not fully understood, but it is generally believed to be released from cells in processes such as apoptosis and necrosis. cfDNA is highly fragmented compared to intact genomic DNA (e.g. see Alcaide et al. (2020) Scientific Reports 10, article 12564), and in general circulates as fragments between 120-220 bp long, with a peak around 168 bp (in humans).

cfDNA is present in many bodily fluids, including but not limited to blood and urine, and the methods and compositions disclosed herein can use any suitable source of cfDNA e.g. a blood sample (such as venous blood) or a urine sample. Ideally cfDNA is isolated from blood, and the blood may be treated to yield plasma (i.e. the liquid remaining after a whole blood sample is subjected to a separation process to remove the blood cells, typically involving centrifugation) or serum (i.e. blood plasma without clotting factors such as fibrinogen). Thus the methods and compositions disclosed herein can be used as part of so-called liquid biopsy testing, and can be implemented using plasma or serum cfDNA. Methods disclosed herein may thus include a step of purifying cfDNA from a blood, plasma or serum sample, to provide cfDNA for digestion and analysis. Methods may also include a step of obtaining a blood sample and preparing plasma or serum therefrom, thus providing a source for downstream purification of cfDNA.

Blood can be collected in tubes that contain an anticoagulant and an agent to inhibit genomic DNA from white blood cells in the sample being released into the plasma component of the blood sample. Such tubes are commercially available as glass cfDNA ‘Blood Collection Tubes’ or ‘BCT’ from Streck (La Vista, NE) e.g. as discussed by Diaz et al. (2016) PLoS One 11(11): e0166354, and they can stabilize cfDNA within blood for up to 14 days at 6-37° C. (thus providing advantages compared to typical K₂EDTA collection tubes). Useful anticoagulants include, but are not limited to, EDTA, heparin, or citrate. Useful agents to inhibit release of genomic DNA from white blood cells include, but are not limited to, diazolidinyl urea, imidazolidinyl urea, dimethoylol-5,5-dimethylhydantoin, dimethylol urea, 2-bromo-2-nitropropane-1,3-diol, oxazolidines, sodium hydroxymethyl glycinate, 5-hydroxy-methoxymethyl-1-laza-3,7-dioxabicyclo[3.3.0]octane, 5-hydroxymethyl-1-1 aza-3,7dioxa-bicyclo[3.3.0]octane, 5-hydroxypoly [methyleneoxy]methyl-1-laza-3,7dioxabicyclo[3.3.0]octane, quaternary adamantine, and mixtures thereof. Other useful components can include a quenching agent (e.g. lysine, ethylene diamine, arginine, urea, adenine, guanine, cytosine, thymine, spermidine, or any combination thereof) which can abate free aldehyde from reacting with DNA within a sample, aurintricarboxylic acid, metabolic inhibitors (e.g. glyceraldehyde and/or sodium fluoride), and/or nuclease inhibitors. For instance, a tube can include imidazolidinyl urea (or diazolidinyl urea), EDTA and glycine. Further information about suitable collection tubes can be found in WO2013/123030 and US2010/0184069.

Other useful collection tubes are available, including but not limited to various plastic tubes: the ‘Cell-Free DNA Collection Tube’ from Roche, made of PET; the ‘LBgard blood tube’ from Biomatrica, made from plastic and suitable for up to 8.5 mL of blood; and the ‘PAXgene Blood DNA tube’ from PreAnalytiX or Qiagen. These various tubes are discussed in more detail in Kerachian et al. (2021) Clinical Epigenetics 13,193, Schmidt et al. (2017) Clinica Chimica Acta 269:94-8, and Grölz et al. (2018) Current Pathobiology Reports 6:275-86.

These various tubes can store up to 8.5 mL of blood, or sometimes up to 10 mL. A blood sample taken from a subject may thus typically have a volume of between 5-10 mL.

A 10 mL blood sample typically yields between 10-500 ng cfDNA, but can sometimes yield substantially higher amounts e.g. up to around 10 μg, particularly in certain cancer patients. Methods disclosed herein can be performed on the amount of cfDNA contained in a 10 mL blood sample. Methods and compositions disclosed herein may typically use from 10-400 ng of cfDNA, for instance from 10-250 ng or from 10-200 ng.

Analysis of plasma-derived cfDNA is preferred. Kits for purifying cfDNA from plasma (and other bodily fluids) are readily available e.g. the MagMAX cfDNA isolation kit from ThermoFisher, the Maxwell RSC ccfDNA plasma kit from Promega, the Apostle MiniMax high efficiency isolation kit from Beckman Coulter, or the QIAamp or EZ1 products from Qiagen.

Methods and compositions disclosed herein may therefore utilise cfDNA extracted from a biological fluid sample of a subject, typically from a plasma or serum sample. Methods may begin with cfDNA which has already been prepared, or may include an upstream step of preparing the cfDNA. Similarly, methods may include an upstream step of obtaining a plasma sample before a step of preparing cfDNA from the plasma sample.

Preferably, the cfDNA utilised in methods and composition disclosed herein is substantially free of single-stranded DNA (ssDNA) i.e. where less than 7% of the cfDNA molecules (by number) are single-stranded, and preferably less than 5% or less than 1% (i.e. such that at least 99% of the cfDNA molecules are double-stranded). In some embodiments, the cfDNA contains less than 0.1% ssDNA, less than 0.01% ssDNA, or may even contain no ssDNA (i.e. free of ssDNA). Extraction of cfDNA to obtain a cfDNA sample substantially free of ssDNA is described, for example, in WO2020/188561.

Ensuring low levels of ssDNA avoids potential inhibition of restriction digestion, and is also useful after digestion as ssDNA can interfere with downstream steps such as ligation and amplification. Commercial kits are available for quantifying single-stranded DNA in a sample e.g. the Promega QuantiFluor™ kit.

In some embodiments, all extracted cfDNA is used in the methods disclosed herein. In other embodiments, cfDNA is split into multiple fractions, and one or more fractions is not used in the methods disclosed herein but may instead be used in other analytical methods, or is kept for use in control experiments, or for other purposes.

In some embodiments, cfDNA is quantified prior to digestion (e.g. by weight, by concentration, etc.). In other embodiments, cfDNA is not quantified prior to digestion.

cfDNA used with the methods and compositions disclosed herein can be obtained from any eukaryotic subject, such as a mammal, and is ideally obtained from a human subject. In some embodiments the human subject may be known or suspected to have a disease (e.g. a cancer). In other embodiments the human subject may be known to be healthy. In some embodiments, the subject is not a pregnant woman.

### Restriction Enzymes and Digestion

Methods and compositions disclosed herein use restriction enzymes which recognise specific sequences in double-stranded DNA and introduce a double-stranded break into the DNA. The enzymes have a recognition site which contains a CpG sequence. Type II restriction enzymes are particularly useful i.e. enzymes where the double-stranded break is introduced within the recognition site. The use of multiple restriction enzymes permits simultaneous digestion in parallel within a sample.

More specifically, methods and compositions disclosed herein use methylation-sensitive restriction enzymes and/or methylation-dependent restriction enzymes. A MSRE cleaves the target DNA only if a CpG within its recognition site is unmethylated, and methylation inhibits the cleavage. Conversely, a MDRE cleaves the target DNA only if a CpG within its recognition site is methylated. MSREs and MDREs are readily available from well-known commercial suppliers, such as ThermoFisher, New England Biolabs, Promega, etc.

MSREs include, but are not limited to: AatII, AccII, AciI, AclI, AfeI, AgeI, Aor13HI, Aor51HI, AscI, AsiSI, Aval, BceAI, BmgBI, BsaAI, BsaHI, BsiEI, BsiWI, BsmBI, BspDI, BspT104I, BssHII, BstBI, BstUI, Cfr10I, ClaI, CpoI, DpnII, EagI, Eco52I, FauI, FseI, FspI, HaeII, HapII, HgaI, HhaI, HinPII, HpaII, Hpy99I, HpyCH4IV, KasI, MluI, NaeI, NarI, NgoMIV, NotI, NruI, NsbI, PaeR7I, PluTI, PmaCI, PmlI, Psp1406I, PvuI, RsrII, SacII, SalI, ScrFI, SfoI, SgrAI, SmaI, SnaBI, SrfI, TspMI, ZraI.

MDREs include, but are not limited to: BspEI, BtgZI, FspEI, GlaI, LpnPI, McrBC, MspJI, XhoI, XmaI.

Methods and compositions disclosed herein can comprise a plurality of restriction enzymes, wherein the plurality consists of MSRE and/or MDRE. Thus the plurality may include only MSREs, only MDREs, or a mixture of both (e.g. one or more MSRE plus one or more MDRE). In general, however, it is preferred to work with MSREs, without needing MDREs, and thus the plurality includes two or more MSREs. Using MSREs leads to cfDNA in which methylated CpG sites are intact but unmethylated CpG sites are digested. Thus, for any particular CpG-containing restriction site in a cfDNA sample, a higher percentage of methylation at this site leads to a lower extent of digestion compared to a cfDNA sample containing a higher percentage of methylation at this site.

A preferred plurality of MSREs includes both HinP1I and AciI. In some embodiments it is possible to use one or more MSREs in addition to HinPII and AciI, but it is more preferred to use HinP1I and AciI as the only two restriction enzymes for digestion of cfDNA. This pairing of enzymes covers over 99% of CpG islands in the human genome. With this MSRE pairing it is preferred to include HinP1I at an excess (measured in terms of enzymatic units) to AciI, and ideally an excess of at least 1.2:1 (i.e. at least 1.2 units of HinP1I for every unit of AciI) e.g. at least 1.5:1, at least 1.75:1, at least 2:1, at least 3:1, at least 4:1, or at least 5:1. Ratios between 2:1 and 5:1 are particularly useful with human cfDNA, and an excess of about 4.5 is preferred e.g. between 4.4 and 4.6. Digestion can be performed at about 37° C., until completion. Incubation at 37° C. for 2 hours is typically adequate for complete digestion of a cfDNA sample using HinPII and AciI as described herein, but longer digestions can be used e.g. when digesting cfDNA obtained from blood which has been stored in a blood collection tube which contains an anticoagulant and an agent to inhibit genomic DNA from white blood cells being released into the plasma component of the blood sample (see elsewhere herein).

In some embodiments HhaI, AspLEI or CfoI is used as one of the restriction enzymes for digestion of cfDNA. Each of HhaI, AspLeI and CfoI is a MSRE and each recognises the same recognition sequence as HinP1I, but does not necessarily cleave at the same cleavage site within the recognition sequence. In some embodiments, SsiI is used as one of the restriction enzymes for digestion of cfDNA. SsiI is a MSRE and recognises the same recognition sequence as AciI, but does not necessarily cleave at the same cleavage site within the recognition sequence. In some embodiments, HinP1I and SsiI are used as the only restriction enzymes for digestion of cfDNA. In some embodiments, AciI and any one of HhaI, AspLEI and CfoI are used as the only restriction enzymes for digestion of cfDNA. Advantageously, AciI and HinP1I can be completely inactivated by heating to 65° C., whereas HhaI and AspLEI are insensitive to heat inactivation. In addition, both AciI and HinP1I use the same optimal buffer for 100% activity, whereas the recommended optimal reaction buffers for each of SsiI, AspLEI and CfoI are different, making it more difficult to tailor the digestion conditions for these alternative enzyme combinations without compromising enzyme activity.

In some embodiments, any combination of MSREs which recognise the same recognition sequence as Hinp1I or AciI, but that do not necessarily cleave at the same cleavage site within the recognition sequence, can be used for digestion of cfDNA.

The concentration of restriction enzymes can be selected according to the particular experiments underway. Typically, HinP1I can be used at 10-450 units per μg cfDNA, and AciI can be used at 2.5-100 units per μg cfDNA e.g. with a ratio of 4.5 units HinP1I per unit of AciI. In other embodiments, HinP1I can be used at 500-2500 units per μg cfDNA, and AciI can be used at 100-500 units per μg cfDNA e.g. with a ratio between 4.4-4.6 units (such as 4.5 units) HinP1I per unit of AciI. In terms of solution concentration, HinP1I can be used at 35-45 units/ml, and AciI can be used at 5-15 units/mL e.g. with a ratio of 4.5 units HinP1I per unit of AciI.

HinP1I recognises the sequence GCGC and cleaves after the first G to leave a two nucleotide 5′ overhang (5′-G/CGC). It cuts well at 37° C. and can be heat-inactivated by heating at 65° C. for 20 minutes. For HinP1I, NEB recommends the use of its rCutSmart™ buffer (50 mM potassium acetate, 20 mM Tris-acetate, 10 mM magnesium acetate, 100 μg/mL recombinant albumin, pH 7.9). 1 unit of HinP1I is defined as the amount of enzyme required to digest 1 μg of λ DNA in 1 hour at 37° C. in a total reaction volume of 50 μl.

Some commercial suppliers provide the Hin6I enzyme instead of HinP1I. These two enzymes have essentially the same properties i.e. they have the same recognition sequence, the same optimum digestion temperature, and they can both be inactivated at 65° C. for 20 minutes. Also, 1 unit of Hin6I is defined in the same way as 1 unit of HinP1I. Thus the terms HinP1I and Hin6I are used interchangeably herein, and any enzyme combination which is disclosed as using HinP1I should be understood as also disclosing that same combination using Hin6I instead e.g. the invention provides combinations of AciI & Hin6I in the same manner as disclosed herein for AciI & HinP1I.

AciI recognises the sequence CCGC and cleaves after the first C to leave a two nucleotide 5′ overhang (5′-C/CGC). It cuts well at 37° C. and can be heat-inactivated by heating at 65° C. for 20 minutes. For AciI, NEB recommends the use of its rCutSmart™ buffer (50 mM potassium acetate, 20 mM Tris-acetate, 10 mM magnesium acetate, 100 μg/mL recombinant albumin, pH 7.9). 1 unit of AciI is defined as the amount of enzyme required to digest 1 μg of 2 DNA in 1 hour at 37° C. in a total reaction volume of 50 μl. Its recognition site is non-palindromic.

λ DNA is a commonly used DNA substrate extracted from bacteriophage lambda (cI857ind 1 Sam 7), being 48502 bp long. It is usually stored in 10 mM Tris-HCl (pH 8.0), 1 mM EDTA, and is widely available from commercial suppliers e.g. from NEB under catalogue number N3011S.

Because HinPII and AciI share essentially the same conditions for digestion and inactivation they make a useful pairing for digesting DNA. In contrast, enzymes such as HpaII, Aval, HaeII and MluI, require heating to 80° C. for inactivation. BstUI and PvuI, and HhaI, are not susceptible to heat inactivation. BstUI cuts optimally at 60° C. and shows only 10% of its full activity at 37° C. PvuI shows only 10% of its full activity in NEB's rCutSmart™ buffer.

After digestion it is preferred to inactivate the restriction enzymes, particularly if downstream amplification steps, such as PCR, will be used. Heat inactivation is particularly suitable, and HinP1I and AciI can both be inactivated by heating the composition at 65° C. for at least 20 minutes e.g. for between 20-60 minutes. Further details about inactivation are given below.

Other useful combinations of enzymes comprise or consist of: (i) HinP1I+AciI+McrBC; (ii) HinP1I+AciI+MspJI; (iii) HinP1I+AciI+HpaII+HpyCH4IV+BstUI; (iv) HinP1I+AciI+HpaII+HpyCH4IV+Aval; (v) MspJI+FspEI; (vi) MspJI+HinP1I+AciI; (vii) MspJI+FspEI+HinP1I+AciI; or (viii) MspJI+FspEI+HinP1I+AciI+HpyCH4IV.

Other useful combinations of enzymes comprise or consist of MSREs which recognise the same recognition sequence as HinPII and/or AciI, but that do not necessarily cleave at the same cleavage site within the recognition sequence, including: (i) AciI+HhaI; (ii) AciI+AspLEI; (iii) AciI+CfoI; (iv) SsiI+HinP1; (v) SsiI+HhaI; (vi) SsiI+AspLEI; (vii) SsiI+CfoI; (viii) SsiI+HinP1I. MspJI shares essentially the same conditions for digestion and inactivation as HinP1I and AciI (e.g. it is active at 37° C. in rCutSmart™, and can be inactivated at 65° C.). This trio of enzymes can provide 85% CpG coverage and 100% CpG island coverage, so it is particularly useful.

Two further useful combinations comprise or consist of: (i) HinP1I+AciI+HpaII; or (ii) HinP1I+AciI+HpaII+HpyCH4IV. For these two combinations, methods and compositions of the invention should use at least one of the following additional features, as discussed elsewhere herein: (a) HinP1I is used at an excess to AciI in terms of enzymatic units; (b) digestion occurs for 11 hours or less; (c) the digested cfDNA is subjected to sequencing.

Other useful combinations of enzymes comprise or consist of MSREs which recognise a recognition sequence that comprises the recognition sequence of HinP1I and/or AciI, including: (i) AciI and one of AfeI, Aor51HI, AscI, BssHII, Paul, HaeII, Eco47III, EheI, FspAI, GIaI, KasI, MteI, NarI, NsbI, PluTI, SfoI, or SgsI; (ii) BsrBI and one of AfeI, Aor51HI, AscI, BssHII, Paul, HaeII, Eco47III, EheI, FspAI, GIaI, KasI, MteI, NarI, NsbI, PluTI, SfoI, or SgsI; (iii) MbiI and one of AfeI, Aor51HI, AscI, BssHII, Paul, HaeII, Eco47III, EheI, FspAI, GIaI, KasI, MteI, NarI, NsbI, PluTI, SfoI, or SgsI; (iv) NotI and one of AfeI, Aor51HI, AscI, BssHII, Paul, HaeII, Eco47III, EheI, FspAI, GIaI, KasI, MteI, NarI, NsbI, PluTI, SfoI, or SgsI; (v) SacII and one of AfeI, Aor51HI, AscI, BssHII, Paul, HaeII, Eco47III, EheI, FspAI, GlaI, KasI, MteI, NarI, NsbI, PluTI, SfoI, or SgsI; (vi) Cfr421 and one of AfeI, Aor51HI, AscI, BssHII, Paul, HaeII, Eco47III, EheI, FspAI, GIaI, KasI, MteI, NarI, NsbI, PluTI, SfoI, or SgsI; (vii) SgrBI and one of AfeI, Aor51HI, AscI, BssHII, Paul, HaeII, Eco47III, EheI, FspAI, GIaI, KasI, MteI, NarI, NsbI, PluTI, SfoI, or SgsI.

Other useful combinations of enzymes comprise or consist of MSREs which recognise a recognition sequence that comprises the recognition sequence of HinP1I and/or AciI, including: (i) HinP1I and one of BsrBI, MbiI, NotI, SacII, Cfr421, or SgrBI; (ii) AfeI and one of BsrBI, MbiI, NotI, SacII, Cfr42I, or SgrBI; (iii) Aor51HI and one of BsrBI, MbiI, NotI, SacII, Cfr42I, or SgrBI; (iv) AscI and one of BsrBI, MbiI, NotI, SacII, Cfr42I, or SgrBI; (v) BssHII and one of BsrBI, MbiI, NotI, SacII, Cfr42I, or SgrBI; (vi) Paul and one of BsrBI, MbiI, NotI, SacII, Cfr42I, or SgrBI; (vii) HaeII and one of BsrBI, MbiI, NotI, SacII, Cfr42I, or SgrBI; (viii) Eco47III and one of BsrBI, MbiI, NotI, SacII, Cfr42I, or SgrBI; (ix) EheI and one of BsrBI, MbiI, NotI, SacII, Cfr42I, or SgrBI; (x) FspAI and one of BsrBI, MbiI, NotI, SacII, Cfr42I, or SgrBI; (xi) GIaI and one of BsrBI, MbiI, NotI, SacII, Cfr42I, or SgrBI; (xii) KasI and one of BsrBI, MbiI, NotI, SacII, Cfr421, or SgrBI; (xiii) MteI and one of BsrBI, MbiI, NotI, SacII, Cfr421, or SgrBI; (xv) Narl and one of BsrBI, MbiI, NotI, SacII, Cfr42I, or SgrBI; (xvi) NsbI and one of BsrBI, MbiI, NotI, SacII, Cfr421, or SgrBI; (xvii) PIutI and one of BsrBI, MbiI, NotI, SacII, Cfr42I, or SgrBI; (xviii) SfoI and one of BsrBI, MbiI, NotI, SacII, Cfr42I, or SgrBI; (ixx) SgsI and one of BsrBI, MbiI, NotI, SacII, Cfr42I, or SgrBI.

Where methods are described herein as involving “digestion”, this term (and also “digesting”, etc.) refers to the mixing of active restriction enzymes with DNA in conditions under which digestion can occur. If there are no recognition sites for the restriction enzyme in question (e.g. because it is a MSRE and all of the recognition sequences are fully methylated) then a step of “digestion” still takes place even though DNA cleavage does not occur.

### Methods

Various methods for digesting cfDNA using a combination of restriction enzymes (e.g. a combination of MSREs) are disclosed herein.

Enzymes and cfDNA are typically incubated for a long enough period for substantially complete digestion to occur i.e. further incubation does not lead to any measurable increase in cfDNA cleavage. For a typical sample, this can be achieved by incubation at 37° C. for 2 hours, but longer digestions can be performed if desired e.g. 3 hours, 4 hours, or longer (e.g. overnight). In some embodiments, digestion is performed for 11 hours or less. Thus, in some embodiments, digestion may be performed for between 2-11 hours e.g. for between 2-10 hours, 2-9 hours, 2-8 hours, or 2-4 hours. In other embodiments (e.g. where a collection tube is used, as discussed herein) digestion may be performed for longer periods e.g. for 12 hours or more.

After digestion has occurred, it is preferred to inactivate the restriction enzymes, particularly if downstream amplification steps will be used. HinP1I and AciI can both be inactivated by heating them to 65° C. e.g. by immersing the reaction mixture in a 65° C. water bath. Digestion reaction mixtures with cfDNA tend to have a low volume such that the temperature of the whole reaction mixture reaches 65° C. very quickly, leading to inactivation of the enzymes. In some embodiments heating at this temperature occurs for longer than 15 minutes, and ideally occurs for at least 20 minutes e.g. for 20-60 minutes. The temperature can exceed 65° C. if desired, but this is not required. This heating step is adequate for complete inactivation of the restriction enzymes i.e. such that the enzymes' digestion activity toward cleavable target cfDNA molecules under the digestion conditions employed prior to heating can no longer be measurably detected.

The invention also provides methods for analysing cfDNA, comprising digestion of cfDNA as discussed above, followed by downstream analytical steps e.g. a step of amplification (such as PCR, and in particular real-time PCR), a step of ligation (such as ligation of sequencing adapters), a step of DNA sequencing, etc. See further below.

The invention also provides methods for assessing methylation status of one or more CpG sites in cfDNA, comprising digestion of cfDNA as discussed above, followed by downstream analytical steps which quantify the degree of digestion at the one or more CpG sites. The degree of digestion may be determined individually for each site, or may be determined in aggregate.

The invention also provides methods for diagnosing the presence of absence of a cancer in a subject, comprising assessing methylation status of one or more CpG sites in cfDNA as discussed above, wherein hypermethylation and/or hypomethylation of the one or more CpG sites is associated with the cancer. In some embodiments, methods include a step of preparing a report in paper or electronic form based on the assessment of the presence or absence of the cancer, and optionally communicating the report to the subject and/or a healthcare provider of the subject.

The invention also provides a method for treating or managing a cancer in a subject, comprising diagnosing the presence of cancer as above, and administering a suitable anti-cancer treatment to the subject. The treatment may comprise one or more of surgical resection, chemotherapy, radiation therapy, immunotherapy, and/or targeted therapy.

Preferred methods do not include a step of bisulfite conversion. Other preferred methods include no step in which chemical changes are made to nucleobases within DNA e.g. no bisulfite conversion, no TAPS conversion, etc. TAPS conversion refers to TET-assisted pyridine borane sequencing.

Preferred methods do not use restriction enzyme isoschizomers, where one of the enzymes recognizes both the methylated and unmethylated forms of the restriction site while the other recognizes only one of these forms.

Preferred methods do not use a mixture of restriction enzymes in which at least one enzyme has a recognition sequence which includes a CpG but which is neither a MSRE or a MDRE i.e. an enzyme which digests regardless of the CpG methylation status.

Some methods do not include a step in which a sample containing purified cfDNA is heated prior to digestion. Other preferred methods do not include such a pre-digestion heating step comprising heating the sample above 40° C., above 50° C., above 60° C., above 70° C., or to 80° C. Other preferred methods do not include a pre-digestion heating step comprising heating the sample at ≥80° C. for ≥20 minutes.

### Compositions

Various compositions comprising a plurality of restriction enzymes (e.g. a plurality of MSREs) are disclosed herein. They are typically aqueous compositions comprising the enzymes in soluble active form, along with other components such as salts, buffers, co-factors, etc.

These compositions can include salts and/or buffers in aqueous solution. For instance, the composition can include 50 mM potassium acetate, 20 mM Tris-acetate, 10 mM magnesium acetate, 100 μg/mL recombinant albumin, pH 7.9 (i.e. the composition of the commercial rCutSmart™ buffer). As an alternative, the composition can include 50 mM Tris-HCl, 10 mM MgCl₂, 100 mM NaCl, 100 μg/mL recombinant albumin, pH 7.9 (i.e. the composition of the commercial NEBuffer™ r3.1 product). pH is measured at 25° C.

The compositions can include cfDNA, in particular when being used for digestion. As discussed above, in some compositions HinP1I is present at 10-450 units per μg cfDNA, and AciI is present at 2.5-100 units per μg cfDNA e.g. with a ratio of 4.5 units HinP1I per unit of AciI. In other embodiments, HinP1I can be used at 500-2500 units per μg cfDNA, and AciI can be used at 100-500 units per μg cfDNA e.g. with a ratio between 4.4-4.6 units (such as 4.5 units) HinP1I per unit of AciI. In terms of solution concentration, HinP1I can be present at 35-45 units/ml, and AciI can be present at 5-15 units/mL cfDNA e.g. with a ratio of 4.5 units HinP1I per unit of AciI.

One useful composition of the invention thus comprises HinPII and AciI (e.g. with an excess of HinP1I, as described herein), potassium acetate, Tris-acetate, magnesium acetate, albumin, pH 7.8-8.0 (and, optionally, cfDNA to be digested). For instance, the composition may comprise from 4-5 units HinP1I, from 0.5-1.5 units AciI, 50 mM potassium acetate, 20 mM Tris-acetate, 10 mM magnesium acetate, 100 μg/mL albumin, pH 7.9, and cfDNA.

The restriction enzymes in the compositions are preferably present in enzymatically active form, as this permits their use to digest cfDNA. After digestion, however, the compositions can be heated (e.g. to 65° C.) to inactivate the enzymes, and so in some embodiments the restriction enzymes are present in heat-inactivated form.

In some embodiments, the compositions can also include PCR reagents e.g. suitable buffer/salt components (if required in addition to buffer/salt which persist after digestion), a DNA polymerase (such as a Taq polymerase), dNTPs, primers, probes, etc.

In some embodiments, the compositions can also include sequencing reagents e.g. one or more of sequencing adapters, DNA ligase (such as T4 ligase), Klenow fragment of DNA polymerase I, an A-tailing enzyme (such as Taq polymerase), a blunt-ending polymerase (such as T4 DNA polymerase), a kinase (such as T4 polynucleotide kinase), etc.

In some embodiments, the compositions can also include control DNA, as discussed below.

As noted above, when a composition includes HinP1I and AciI then HinP1I is ideally present at an excess (measured in terms of enzymatic units) to AciI, and ideally an excess of at least 1.2:1 e.g. at least 1.5:1, at least 1.75:1, at least 2:1, at least 3:1, at least 4:1, or at least 5:1. A ratio of at least 2:1 is often useful e.g. when the intention is to analyse human cfDNA, and a ratio of about 4.5:1 has been found to be useful when digesting human cfDNA from plasma.

Preferred compositions do not include restriction enzyme isoschizomers, where one enzyme recognizes both the methylated and unmethylated forms of a restriction site and another recognizes only one of these forms.

Preferred compositions do not include a mixture of restriction enzymes in which at least one enzyme has a recognition sequence which includes a CpG but which is neither a MSRE or a MDRE i.e. an enzyme which digests regardless of the CpG methylation status.

### Downstream Amplification

After digestion, methods disclosed herein may include a step of amplification (e.g. PCR) performed on the digested cfDNA. Typically this amplification will be targeted to one or (preferably) more loci of interest e.g. loci containing CpG sites whose methylation status is known or expected to be associated with a particular biological state (e.g. with a cancer of interest). Thus upstream and downstream primers are used which flank the CpG site of interest, and the intervening CpG-containing sequence will be amplified if it has not been digested by restriction enzymes. The resulting amplicons can then be detected e.g. using a labelled probe which is complementary to a sub-sequence within the amplicons of interest.

Methods may therefore include a step of adding PCR reagents after digestion e.g. suitable buffer/salt components (if required in addition to buffer/salt remaining from digestion), a DNA polymerase (such as a Taq polymerase), dNTPs, primers and (optionally) probes. As an alternative, one or more of these components may be present during digestion e.g. it is possible to use a hot start PCR protocol, such that PCR reagents are already present during the digestion step but they do not become active until the reaction mixture is heated (e.g. during heat inactivation of the restriction enzymes).

Restriction digestion typically takes place in the presence of high levels of Mg⁺⁺. PCR usually relies on Mg⁺⁺, so standard PCR buffers include Mg⁺⁺. In this situation, however, addition of a standard PCR buffer can lead to an excess of Mg⁺⁺ which can inhibit efficiency of amplification. Thus added PCR reagents may include a lower level of Mg⁺⁺ than would normally be the case.

Where PCR primers and probes are present during MSRE digestion, they should be designed so that their sequences do not include the recognition site for the MSRE(s) which is/are being used. Amplification and detection of amplicons may be carried out by conventional PCR using fluorescently-labeled primers followed by capillary electrophoresis of amplification products. In some embodiments, following amplification the amplification products are separated by capillary electrophoresis and fluorescent signals are quantified. An electropherogram plotting the change in fluorescent signals as a function of size (bp) or time from injection may be generated, wherein each peak in the electropherogram corresponds to the amplification product of a single locus. The peak's height (provided for example using “relative fluorescent units”, rFU) may represent the intensity of the signal from the amplified locus. Computer software may be used to detect peaks and calculate the fluorescence intensities (peak heights) of a set of loci whose amplification products were run on the capillary electrophoresis machine, and subsequently the ratios between the signal intensities.

A preferred PCR technique is real-time PCR (also known as qPCR), in which simultaneous amplification and detection of the amplification products are performed. Real-time PCR can be used with non-specific detection or sequence-specific detection. Non-specific detection (e.g. using a dsDNA-binding dye, such as SYBR Green) can be used within the methods disclosed herein, but is not ideal if it is desired to distinguish between multiple different amplicons in the same reaction. Thus it is more typical to use sequence-specific detection, and methods and compositions may use a labelled oligonucleotide probe (usually with a fluorophore and fluorescence quencher on the same probe, as in the TaqMan system) which is complementary to a specific sequence within nucleic acid amplicon(s) of interest. Different probes for amplicons derived from different target CpGs can be labelled with different fluorophores so that multiple different amplicons can be distinguished.

Real-time PCR may thus be achieved by using a hydrolysis probe based on combined reporter and quencher molecules. In such assays, oligonucleotide probes have a fluorescent moiety (fluorophore) attached to their 5′ end and a quencher attached to the 3′ end. During PCR amplification, the polynucleotide probes selectively hybridize to their target sequences on the template, and as the polymerase replicates the template it also cleaves the polynucleotide probes due to the polymerase's 5′-nuclease activity. When the polynucleotide probes are intact, the close proximity between the quencher and the fluorescent moiety normally results in a low level of background fluorescence. When the polynucleotide probes are cleaved, the quencher is decoupled from the fluorescent moiety, resulting in an increase of intensity of fluorescence. The fluorescent signal correlates with the amount of amplification products, i.e. the signal increases as the amplification products accumulate.

Suitable fluorophores include, but are not limited to, fluorescein, FAM, lissamine, phycoerythrin, rhodamine, Cy2, Cy3, Cy3.5, Cy5, Cy5.5, Cy7, FluorX, JOE, HEX, NED, VIC and ROX. Suitable fluorophore/quencher pairs are known in the art, including but not limited to: FAM-TAMRA, FAM-BHQ1, Yakima Yellow-BHQ1, ATTO550-BHQ2 and ROX-BHQ2.

Fluorescence may be monitored during each PCR cycle, providing an amplification plot showing the change of fluorescent signals from the probe(s) as a function of cycle number. In the context of real-time PCR, the following terminology is used:

“Quantification cycle” (“Cq”) refers to the cycle number in which fluorescence increases above a threshold, set automatically by software or manually by the user. In some embodiments, the threshold may be constant for each CpG locus of interest and may be set in advance, prior to carrying out the amplification and detection. In other embodiments, the threshold may be defined separately for each CpG locus after the run, based on the maximum fluorescence level detected for this locus during the amplification cycles.

“Threshold” refers to a value of fluorescence used for Cq determination. In some embodiments, the threshold value may be a value above baseline fluorescence, and/or above background noise, and within the exponential growth phase of the amplification plot.

“Baseline” refers to the initial cycles of PCR where there is little to no change in fluorescence. Computer software is readily available for analysing amplification plots and determining baseline, threshold and Cq.

Where a CpG site has not been digested, and is thus amplified in subsequent PCR, relatively low Cq values are seen because detectable amplification products accumulate after a relatively small number of amplification cycles. Conversely, if amplicons are present at lower levels (e.g. because some CpG loci of interest were digested) then fewer amplicons are seen, and the Cq value is higher.

These results can thus indicate, for any given CpG site, the proportion of cfDNA molecules in a sample which were methylated/unmethylated at that CpG site. These FIGURES can be expressed as a percentage, a fraction, a normalised value, etc.

Primers may vary in length, depending on the particular assay format and the particular needs. In some embodiments, the primers may be at least 15 nucleotides long, such as between 15-25 nucleotides or 18-25 nucleotides long. The primers may be adapted to be suited to a chosen amplification system.

Primers may be designed to generate amplicons between 60-150 bp long (when the relevant CpG site(s) is/are intact) e.g. between 70-140 bp long.

Oligonucleotide probes may vary in length. In some embodiments, the probes may include between 15-30 nucleotides, from 20-30 nucleotides, or from 25-30 nucleotides.

The oligonucleotide probes may be designed to bind to either strand of the double-stranded amplicons. Additional considerations include the melting temperature of the probes, which should preferably be comparable to that of the primers.

Where multiple CpG sites are analysed in parallel, with simultaneous amplification of more than one target in the same reaction mixture (co-amplification) using different primer pairs for each CpG site of interest, these different primers may be designed such that they can work at the same annealing temperature during amplification. Thus primers with similar melting temperature (Tm) can be designed e.g. within ±3°-5° C. of each other. Similar considerations apply where multiple probes are used.

Computer software is readily available for routine designing of primers and probes which meet the various requirements of any particular experiment.

### Downstream Sequencing

After digestion, methods disclosed herein may include a step of DNA sequencing, such as a step using next-generation sequencing (‘NGS’) techniques (also known as high-throughput sequencing). NGS generally involves three basic steps: library preparation; sequencing; and data processing. Examples of NGS techniques include sequencing-by-synthesis and sequencing-by-ligation (employed, for example, by Illumina Inc., Life Technologies Inc., PacBio, and Roche), nanopore sequencing methods and electronic detection-based methods such as Ion Torrent™ technology (Life Technologies Inc.). NGS may be performed using various high-throughput sequencing instruments and platforms, including but not limited to: Novaseq™, Nextseq™ and MiSeq™ (Illumina), 454 Sequencing (Roche), Ion Chef™ (ThermoFisher), SOLID® (ThermoFisher) and Sequel II™ (Pacific Biosciences). Appropriate platform-designed sequencing adapters are used for preparing the sequencing library, and are readily available from the platforms' manufacturers.

Library preparation for the major high-throughput sequencing platforms involves ligation of specific adapter oligonucleotides, also termed “sequencing adapters”, to the DNA fragments to be sequenced. Sequencing adapters typically include platform-specific sequences for fragment recognition by a particular sequencer e.g. sequences that enable ligated molecules to bind to the flow cells of Illumina platforms (e.g. the P5 and P7 sequences). Each sequencing instrument provider typically sells a specific set of sequences for this purpose. Further details of library preparation are discussed below.

Sequencing adapters can include sites for binding to a universal set of PCR primers. This permits multiple adapter-ligated DNA molecules to be amplified in parallel by PCR, using a single set of primers.

Sequencing adapters can include sample indices, which are sequences that enable multiple samples to be combined, and then sequenced together (i.e. multiplexed) on the same instrument flow cell or chip. Each sample index, typically 6-10 nucleotides, is specific to a given sample and is used for de-multiplexing during downstream data analysis to assign individual sequence reads to the correct sample. Sequencing adapters may contain single or dual sample indexes depending on the number of libraries combined and the level of accuracy desired.

Sequencing adapters can include unique molecular identifiers (UMIs) to provide molecular tracking, error correction and increased accuracy during sequencing. UMIs are short sequences, typically 5 to 20 bases in length, used to uniquely identify original molecules in a sample library. As each nucleic acid in the starting material is tagged to provide a unique molecular barcode, bioinformatics software can filter out duplicate reads and PCR errors with a high level of accuracy and report unique reads, removing the identified errors before final data analysis.

In some embodiments, sequencing adapters include both a sample barcode sequence and a UMI.

In some embodiments, sequencing adapters allow for paired-end sequencing.

In some embodiments, the compositions and methods disclosed herein use Y-shaped sequencing adapters i.e. adapters consisting of two single-stranded oligonucleotides which anneal to provide a double-stranded stem and two single-stranded ‘arms’. In other embodiments, the compositions and methods disclosed herein use hairpin sequencing adapters i.e. a single-stranded oligonucleotide whose 5′ and 3′ termini anneal to provide a double-stranded stem. For both Y-shaped and hairpin adapters the double-stranded stem can include a short single-stranded overhang e.g. a single A or T nucleotide. For both Y-shaped and hairpin adapters the double-stranded stem can be ligated to a cfDNA fragment, to prepare a sequencing library.

Suitable sequencing adapters for use in the compositions and methods disclosed herein may thus be TruSeq™ or AmpliSeq™ or TruSight™ adapters (for use on the Illumina platform) or SMRTbell™ adapters (for use on the PacBio platform).

Where sequencing adapters are added by ligation, this usually occurs at both ends of the DNA to be sequenced.

Restriction digestion can leave blunt-ends, but typically produces a single-stranded overhang. Library preparation steps can either preserve this overhang (i.e. add complementary nucleotides) or remove it. As the sequence of a post-digestion terminal single-stranded overhang can include useful information then it is preferred to add sequencing adapters in a way which preserves the overhang e.g. using enzymatic ligation in which a ligase enzyme covalently links a sequencing adapter to a DNA fragment where the terminal sequence of the adapter is complementary to the terminal sequence obtained using the restriction enzyme, or by using a polymerase to add complementary nucleotides and generate a blunt-ended fragment.

In addition to removing or filling in single-strand overhangs, end repair methods carried out before adapter ligation can ensure that DNA molecules contain 5′ phosphate and 3′ hydroxyl groups.

For some libraries, incorporation of a non-templated deoxyadenosine 5′-monophosphate (dAMP) onto the 3′ end of blunted DNA fragments is used in library preparation (a process known as dA-tailing). dA-tails prevent concatemer formation during downstream ligation steps and enable DNA fragments to be ligated to adapter oligonucleotides with complementary dT-overhangs.

As noted above, restriction digestion typically takes place in the presence of high levels of Mg⁺⁺. Sequencing library preparation may also rely on Mg⁺⁺, so standard library prep buffers include Mg⁺⁺. In this situation, however, addition of a standard library prep buffer can lead to an excess of Mg⁺⁺ which can inhibit efficiency of downstream steps. Thus added reagents may include a lower level of Mg⁺⁺ than would normally be the case for library preparation.

As an alternative approach to using lower levels of Mg⁺⁺, it is possible to add a chelating agent after digestion, which can remove the need for removal or dilution of excess Mg⁺⁺ for downstream amplification step(s). It has been found that the addition of a chelating agent at the concentrations disclosed herein impairs neither such amplification step(s) nor subsequent sequencing. The chelating agent can be added to provide an amplification reaction mix comprising the chelating agent and a divalent cation at a molar ratio of between 1:20 to 2:1. For instance, the reaction mix may include 8-20 mM Mg⁺⁺ e.g. about 10 mM magnesium. For instance, amplification may be carried out in a reaction mix comprising between 3-4 mM chelating agent and 4 mM Mg⁺⁺. The chelating agent may comprise one or both of EDTA and EGTA.

After library preparation, the prepared DNA molecules can be sequenced, to provide a plurality of “sequence reads”. These sequence reads are then subjected to data processing e.g. to remove sequences which do not fulfil desired quality criteria, to remove duplicates, to correct sequencing errors, to map sequences onto a reference genome, to count the number of sequence reads, etc. Computer software is readily available for performing these steps.

Any particular CpG site can feature in multiple sequence reads, which can be sequence reads derived from the same original cfDNA molecule and/or from different cfDNA molecules which span the same CpG site. Sequencing is suitably performed such that CpG site(s) of interest is/are seen in at least 100 sequence reads e.g. in at least 200, 300, 400, 500, 600, 700 or more sequence reads. The number of sequence reads that span a particular genomic locus (e.g. a particular CpG site) is referred to as sequencing “depth” or “coverage”. The average number of sequence reads that span a particular genomic locus (e.g. a particular CpG site) is referred to as the average sequencing “depth” or “coverage”. The term “average” refers to the arithmetic mean. Thus, the average depth can be calculated by dividing the sum of nucleotides in sequence reads which map to the human genome by the length of the (haploid) human genome.

Higher sequencing depth increases accuracy by allowing signal to be better distinguished from noise. This is because high-throughput DNA sequencing is error prone (with approximately 0.1-10% of all called bases being incorrect), so sequence reads mapped to a particular locus will often contain (apparent) mutations compared to the reference sequence at that site and/or the other reads mapped to that site.

High depth is useful for determining whether differences in the sequence reads with respect to the reference sequence reflect the underlying sequence of the sample DNA (signal) or are due to errors during sequencing (noise). High depth therefore aids in drawing meaningful conclusions from sequencing data, particularly regarding the presence of rare signals, such as those that result from tumor DNA. Methylated cfDNA molecules from a tumor may be present in blood plasma at amounts in the order of ≤1% of the total cfDNA. Bisulfite sequencing does not provide sufficient depth across a large enough number of genomic sites to reliably detect these rare signals. In contrast, the methods of the invention interrogate CpG sites in the whole human genome with very high average depths, so allowing the detection of these rare signals.

Sequence reads can be mapped to a reference genome i.e. a previously identified genome sequence, whether partial or complete, assembled as a representative example of a species or subject. A reference genome is typically haploid, and typically does not represent the genome of a single individual of the species but rather is a mosaic of the genomes of several individuals. A reference genome for the methods of the present invention is typically a human reference genome e.g. a complete human genome, such as the human genome assemblies available at the website of the National Center for Biotechnology Information or at the University of California, Santa Cruz, Genome Browser. An example of a suitable reference genome for human studies is the ‘hg18’ genome assembly. As an alternative, the more recent GRCh38 major assembly can be used (up to patch p13).

Mapping aligns sequence reads to the reference genome, to identify the location of the reads within the reference genome. The sequence reads that align are designated as being “mapped”. The alignment process aims to maximize the possibility for obtaining regions of sequence identity across the various sequences in the alignment, allowing mismatches, indels and/or clipping of some short fragments on the two ends of the reads. The number of sequence reads mapped to a certain genomic locus is referred to as the “read count” or “copy number” of this genomic locus. It is not necessary to map all sequence reads which are obtained; indeed, it is not unusual that a portion of sequence reads obtained in any given experiment will not be mappable.

The term “genomic locus” refers to a specific location within the genome, and may include a single position (a single nucleotide at a defined position in the genome) or a stretch of nucleotides starting and ending at defined positions in the genome. The specific position(s) may be identified by the molecular location, namely, by the chromosome and the numbers of the starting and ending base pairs on the chromosome. A genomic locus of interest herein contains at least one CpG site.

Where restriction digestion used a MSRE, sequence reads which span a particular CpG site are derived from molecules which were not digested i.e. which (with complete digestion) were methylated at that CpG site. The methylation level of this CpG site can be calculated by dividing its read count by an expected read count of this site (e.g. the read count which would be expected if it was fully methylated, and thus undigested). The expected read count may be determined using, for instance: (i) the read count of a control locus that is not cut by the restriction endonuclease; (ii) the average read count of a plurality such control loci; or (iii) the read count of the same CpG site in an undigested control sample, optionally corrected for sequencing depth differences.

As an alternative, the expected read count for a CpG site may be determined as the sum of the read count at this CpG site (indicating methylation) plus the sum of the read counts whose termini map to this CpG site (indicating non-methylation), taking account where necessary of any end-repair which took place during library preparation.

To avoid double-counting, the non-methylated CpG sites can be taken as sequencing reads whose 5′ ends map to a site, as sequencing reads whose 3′ ends map to a site, or as the half of the sum of sequencing reads whose 5′ ends or 3′ ends map to a site. As some library preparation methods can result in depletion of small fragments, which are then not sequenced (e.g. in CpG islands, where a starting cfDNA molecule is cleaved by a MSRE at more than one unmethylated site, thus providing 3 or more restriction fragments, some of which are very small), the observed number of unmethylated CpG sites may be lower than the true value in the original sample. This distortion can be somewhat addressed by using the larger of the number of reads whose 3′ ends map to a site and the number of reads whose 5′ ends map to a site (or to use the mean).

These calculations can thus provide, for any given CpG site, the proportion of cfDNA molecules in a sample which were methylated at that CpG site. Conversely, similar calculations can provide the proportion of a particular CpG site which were unmethylated. These FIGURES can be expressed as a percentage, a fraction, a normalised value, etc.

One way of expressing coverage of a particular CpG site is referred to as ‘Hitspan100’, which refers to the number of sequence reads which span a certain CpG position with at least 50 nucleotides both upstream and downstream. For example, a Hitspan100 of 90 at a specific CpG site means that there are 90 sequence reads which span this site with at least 50 nucleotides both upstream and downstream.

Methods disclosed herein do not require differential adapter tagging of methylated vs. unmethylated DNA molecules. The same population of adapters can be used for all molecules.

### Controls

Methods disclosed herein can take advantage of positive and negative controls. In some embodiments, parallel analysis can be performed on one or more of:
DNA control which does not contain a recognition sequence for the restriction enzymes used for digestion. If this DNA is digested, this indicates that the method has not performed correctly.
DNA control which contains a fully methylated recognition sequence for the restriction enzymes used for digestion. If this DNA is digested when a method uses only MSREs, this indicates that the method has not performed correctly (and conversely for MDREs).
DNA control which contains a fully unmethylated recognition sequence for the restriction enzymes used for digestion. If this DNA is not fully digested when a method uses only MSREs, this indicates that the method has not performed correctly (and conversely for MDREs).

These DNA controls can also be used as a reference point for analysis, for checking completeness of digestion, etc. As mentioned above, for instance, if fragments are obtained using MSRE digestion then it can be useful in a downstream NGS experiment to know the expected read count, and one way of obtaining this value is to look at the read count for DNA which does not contain the recognition sequence for the MSRE, or at the read count for DNA which contains the recognition sequence but is fully methylated.

For these purposes, it is preferred that the DNA control should be similar in size and composition to cfDNA molecules which contain CpG sites of interest. Thus, although it is possible to use synthetic DNA or PCR amplicons or bacterial plasmid DNA as an unmethylated control, these are more useful if they have sizes which are similar to cfDNA (e.g. a long synthetic DNA, or an appropriately-sized restriction fragment prepared from a plasmid).

Control experiments can be performed internally in a sample, or externally. For an internal control, control DNA can be present in a sample already (e.g. cfDNA containing a CpG site which is known to be ubiquitously (un)methylated, or cfDNA which does not contain a recognition sequence for the restriction enzymes being used) and/or can be added (e.g. synthetic DNA, added to cfDNA). The control DNA can therefore be processed in combination with the cfDNA, and experiences the same conditions as the cfDNA, and so a method can involve co-amplification of a restriction locus and a control locus. For an external control, control DNA is subjected to the same treatment as the cfDNA but not as part of the same reaction mixture.

Thus control DNA, like cfDNA, can be digested with restriction enzymes and then subjected to downstream analytical steps e.g. amplification, DNA sequencing, etc. Real-time PCR of suitable control loci can give a result that can be used as a reference point. For instance, the signals obtained from cfDNA at a CpG site of interest and from control DNA (in particular, from control DNA which is not digested by the restriction enzymes being used) can be compared, and the signal ratio can be used to determine the degree of methylation at a CpG site of interest, because the ratio of signal reflects the ratio of methylation. Thus methods disclosed herein can be performed without requiring evaluation of absolute methylation levels at genomic loci, but rather by calculating a signal ratio between the analyzed genomic loci and a control. This contrasts with some conventional methods of methylation analysis for distinguishing between tumor-derived and normal DNA, which require determining actual methylation levels at specific genomic loci. The methods disclosed herein can thus eliminate the need for standard curves and/or additional laborious steps involved in determination of absolute methylation levels, thereby offering a simple and cost-effective procedure. An additional advantage when using an internal control is that signal ratios are obtained for loci amplified in the same reaction mixture under the same reaction conditions, which can help to eliminate sources of potential error (e.g. the potential for differences between reaction mixtures, such as the concentration of template, enzyme, etc.).

Methods which use qPCR may therefore involve calculating signal intensity ratios between a CpG site co-amplified after digestion of DNA as disclosed herein, thereby providing a methylation status for the CpG site. This methylation status can then be compared to reference values (e.g. obtained from healthy subjects, or from subjects having a known disease) and, based on the comparison, a diagnostic result can be derived. Thus a method may involve: co-amplifying from restriction endonuclease-digested DNA a CpG site and a control locus, thereby generating co-amplification products; determining a signal intensity for each generated co-amplification product; and calculating a ratio between the signal intensities of the co-amplification products of the CpG site and the control locus.

The ratio between the signal intensities of the co-amplification products may be calculated by determining the quantification cycle (Cq) for each locus and calculating 2 (Cq control locus-Cq CpG site) In other words, the reduction in Cq relative to the control locus is determined, and this value is used as the exponent of 2 to calculate the ratio.

Thus, using qPCR or sequencing, it is possible, based on the degree of digestion at any particular CpG site, to derive a numerical value which represents the degree of methylation of that CpG site in a cfDNA sample. This value may be expressed in a variety of ways e.g. as a ratio or percentage of the cfDNA molecules that are methylated at a CpG site, or as an intensity of a signal obtained from a particular CpG site, or as the ratio between a CpG site and a control locus, etc.

### Systems and Kits

The invention also provides various systems and kits.

A system can comprise computer processor(s) for performing and/or controlling the methods disclosed herein, and/or for processing the results e.g., for performing calculations based on the results. Methods which are at least partially computer-implemented are provided.

A system or kit may comprise: a blood, plasma or serum sample of a human subject; components for carrying out a method disclosed herein on at least one CpG site; and computer software stored on a non-transitory computer readable medium, the computer software being able to direct a computer processor to determine a methylation value for the at least one CpG locus based on the methylation assay. The software may also be able to link the methylation value to a diagnostic result or prediction e.g. by comparing one or more methylation value(s) to one or more reference values to assess the presence of a disease in the subject. The computer software may receive data from a qPCR and/or a NGS experiment.

Components for carrying out a method disclosed herein encompass biochemical components (e.g., enzymes, primers, probes, NTPs, etc.), chemical components (e.g., buffers, reagents), and technical components (e.g., a PCR system, such as a real-time PCR system, and equipment such as tubes, vials, plates, pipettes).

The system may be able to prepare and/or communicate a report to the subject and/or to a healthcare provider of the subject, based on the methylation values.

Computer software includes processor-executable instructions that are stored on a non-transitory computer readable medium. The computer software may also include stored data. The computer readable medium is a tangible computer readable medium, such as a compact disc (CD), magnetic storage, optical storage, random access memory (RAM), read only memory (ROM), or any other tangible medium.

Computer-related methods and steps described herein are implemented using software stored on non-volatile or non-transitory computer readable instructions that when executed configure or direct a computer processor or computer to perform the instructions.

Each of the system, server, computing device, and computer described in this application can be implemented on one or more computer systems and be configured to communicate over a network.

They all may also be implemented on one single computer system. In one embodiment, the computer system includes a bus or other communication mechanism for communicating information, and a hardware processor coupled with bus for processing information.

A computer system also includes a main memory, such as a random-access memory (RAM) or other dynamic storage device, coupled to bus for storing information and instructions to be executed by processor. Main memory also may be used for storing temporary variables or other intermediate information during execution of instructions to be executed by processor. Such instructions, when stored in non-transitory storage media accessible to processor, render computer system into a special-purpose machine that is customized to perform the operations specified in the instructions.

A computer system can include read only memory (ROM) or other static storage device coupled to bus for storing static information and instructions for processor. A storage device, such as a magnetic disk or optical disk, is provided and coupled to bus for storing information and instructions.

A computer system may be coupled via bus to a display, for displaying information to a computer user.

An input device, including alphanumeric and other keys, can be coupled to bus for communicating information and command selections to processor. Another type of user input device is cursor control, such as a mouse, a trackball, or cursor direction keys for communicating direction information and command selections to processor and for controlling cursor movement on display.

Methods disclosed herein may be performed by a computer system in response to the processor executing one or more sequences of one or more instructions contained in main memory. Such instructions may be read into main memory from another storage medium, such as storage device. Execution of the sequences of instructions contained in main memory causes the processor to perform the process steps described herein. In alternative embodiments, hard-wired circuitry may be used in place of or in combination with software instructions.

Suitable storage media include any non-transitory media that store data and/or instructions that cause a machine to operation in a specific fashion. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media are distinct from, but may be used in conjunction with, transmission media. Transmission media participates in transferring information between storage media. For example, transmission media includes coaxial cables, copper wire and fiber optics, including the wires that comprise bus.

The invention also provides a kit comprising: (i) a composition comprising a plurality of restriction enzymes, as discussed above; and (ii) components for analysing cfDNA which has been digested with the composition. These components may be e.g. components for performing PCR, or for preparing a sequencing library from digested cfDNA. For instance, the kit may include one or more of: (a) a buffer solution e.g. with 50 mM potassium acetate, 20 mM Tris-acetate, 10 mM magnesium acetate, 100 μg/mL recombinant albumin, pH 7.9, or with 50 mM Tris-HCl, 10 mM MgCl₂, 100 mM NaCl, 100 μg/mL recombinant albumin, pH 7.9; (b) a DNA polymerase, dNTPs, primers and, optionally, one or more probes; (c) sequencing adapters; (d) an enzyme solution, including a DNA ligase and/or a DNA polymerase; and/or (e) control DNA Further details of these components (a) to (e) are discussed elsewhere herein.

A kit may include an instruction manual for carrying out the methods as disclosed herein.

A kit may include a non-transitory computer readable medium storing a computer software comprising instructions that when executed configure or direct a computer processor to perform the method steps disclosed herein.

### Disclaimers

In some instances, the disclosure of WO2022/107145 (PCT/IL2021/051382) is excluded.

In some embodiments, compositions and methods disclosed herein do not use a mixture of cfDNA from 50-60 people.

In some embodiments, compositions and methods disclosed herein do not use between 760-810 ng of cfDNA (in particular, between 760-810 ng of cfDNA from healthy patients).

In some embodiments, compositions and methods disclosed herein do not use 26 ng or 94 ng of cfDNA from treatment-naïve non-small cell lung cancer patients.

In some embodiments, compositions and methods disclosed herein do not use from 25-95 ng of cfDNA from treatment-naïve non-small cell lung cancer patients.

In some embodiments, compositions and methods disclosed herein do not use a panel consisting of 8 CpG sites located in the hg18 human genome assembly at positions chr1-11397653, chr17-17362652, chr17-71690026, chr3-121760779, chr12-49705230, chr1-8120128, chr2-39309230, and chr12-84283776 (as disclosed in Table 4 of PCT/IL2021/051382).

In some embodiments, compositions and methods disclosed herein do not use a mixture of 10 units HinP1I and 5 units AciI.

In some embodiments, compositions and methods disclosed herein do not use a 2:1 activity ratio of HinP1I:AciI.

### General

The practice of the present invention will employ, unless otherwise indicated, conventional methods of chemistry, biochemistry, and molecular biology, within the skill of the art. Such techniques are explained fully in the literature. See, e.g., Methods In Enzymology (Academic Press, Inc.), Green & Sambrook (2012) Molecular Cloning: A Laboratory Manual, 4th edition (Cold Spring Harbor Press), Ausubel et al. (eds) Short protocols in molecular biology, 5th edition (Current Protocols), Molecular Biology Techniques: An Intensive Laboratory Course, (Ream & Field, eds., 1998, Academic Press), Wilson and Walker's Principles and Techniques of Biochemistry and Molecular Biology (Hodmann & Clokie, 2018), Basic Molecular Biology & Techniques-Recent Advances: Molecular Biology & Its Technique (Singh et al., 2021), etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

The term “between” with reference to two values includes those two values e.g. the range “between” 10 mg and 20 mg encompasses inter alia 10, 15, and 20 mg.

Unless specifically stated, a method comprising a step of mixing two or more components does not require any specific order of mixing. Thus components can be mixed in any order. Where there are three components then two components can be combined with each other, and then the combination may be combined with the third component, etc.

The various steps of methods may be carried out at the same or different times, in the same or different geographical locations, e.g. countries, and by the same or different people or entities.

### EXAMPLES

The human genome sequence was analysed for the presence of the recognition sequences of various MSRE and MDRE. The proportion of total CpG sites in the genome (around 28 million) which is accessible to various different MSRE and MDRE is represented in Table 1 and is as follows:

TABLE 1

| | CpG | | CpG |
|---|---|---|---|
| MSRE | coverage | MDRE | coverage |
| AciI/SsiI | 14.56% | MspJI | 79.65% |
| HpaII/HapII | 8.14% | McrBC | 56% |
| HpyCH4IV/HpySE526I / | 7.63% | FspEI | 28% |
| MaeII/TaiI | | | |
| HinP1I/HhaI/CfoI/AspLEI | 5.87% | LpnPI | 8.14% |
| BstUI/AccII/Bsh1236I | 4.97% | | |
| AvaI | 3.54% | | |
| BsaAI | 2.46% | | |
| BmgBI | 0.95% | | |
| AcII | 0.58% | | |
| SnaBI | 0.45% | | |
| BstBI | 0.37% | | |
| ZraI | 0.25% | | |
| PvuI | 0.09% | | |
| HaeII | 3.91% | | |
| MIuI | 0.15% | | |
| PmII | 1.97% | | |

CpG coverage increases when combinations of enzymes are used, and these combinations can provide a recognition site in more than 99% of CpG islands in the human genome, as shown in Table 2:

TABLE 2

| | CpG | CpG island |
|---|---|---|
| Combination | coverage | coverage |
| HpyCH4IV | 8% | 63% |
| HinP1I/HhaI/CfoI/AspLEI | 6% | 94% |
| HpaII/HapII/MspI | 8% | 95% |
| AciI/SsiI | 15% | 98% |
| AvaI | 3.5% | 79% |
| BstUI/AccII/Bsh1236I | 5% | 91.84% |
| HhaI + HpaII | 14% | 98% |
| HhaI + BstUI | 9% | 96.94% |
| HhaI + HinP1I | 6% | 94% |
| HinP1I + AvaI | 10% | 96% |
| HinP1I + HpaII | 14% | 98% |
| HpaII + AciI | 22% | 99% |
| HpyCH4IV + AciI | 22% | 99% |
| HinP1I + AciI | 20% | 99.24% |
| HpaII + CfoI | 14% | 98.51% |
| HpaII + HpyCH4IV | 16% | 97.76% |
| BstUI + MIuI | 5% | 91.84% |
| BstUI + HpaII | 13% | 98.44% |
| HpaII + PmII | 9% | 96.28% |
| HhaI + Bsh12361 | 9% | 96.94% |
| AccII + HpaII | 13% | 98.44% |
| AccII + HpyCH4IV* | ≤13% | |
| HinP1I + AciI + HpaII | 29% | 99.57% |
| HpaII + BstUI + HinP1I* | ≤19% | |
| HinP1I + AciI + McrBC | 59% | 99.99% |
| HinP1I + AciI + MspJI | 85% | 100% |
| BstUI + HhaI + HpaII* | ≤19% | |
| AccII + HpaII + HpyCH4IV* | ≤21% | |
| Bsh12361 + HpaII + HinP1I* | ≤19% | |
| HinP1I + AciI + HpaII + HpyCH4IV | 36% | 99.74% |
| HinP1I + AciI + HpaII + HpyCH4IV + BstUI | 38% | 99.77% |
| HinP1I + AciI + HpaII + HpyCH4IV + AvaI | 38% | 99.78% |
| HinP1I + HpaII + AciI + BstUI + HhaI* | ≤34% | |
| HpaII + HinP1I + AciI + HpyCH4IV | 36% | 99.74% |
| MspJI | 80% | 99.99% |
| MspJI + FspEI | 84% | 100% |
| MspJI + HinP1I + AciI | 85% | 100% |
| MspJI + FspEI + HinP1I + AciI | 88% | 100% |
| MspJI + FspEI + HinP1I + AciI + HpyCH4IV | 89% | 100% |

*Enzyme combinations denoted with (*) represent enzyme combinations in which the CpG coverage has been calculated based on the sum of the number of recognition sequences that exist within the human genome (hg18 genomic build) for each enzyme. In instances where the recognition sequence of one enzyme overlaps with the recognition sequence of another enzyme, and the same CpG site is encompassed by each recognition sequence, an overestimation of the CpG coverage is calculated. The calculated CpG coverage for such enzyme combinations represents the maximal CpG coverage (i.e., if there is no overlap between recognition sequences across the whole human genome). Maximal CpG coverage is indicated by “≤” in Table 2 above, e.g. “≤13%”. For enzyme combinations that are not denoted with (*), the overlap in recognition sequence has been taken into account when calculating the CpG coverage/CpG island coverage and therefore the relevant values in Table 2 above represent the absolute CpG coverage/CpG island coverage values.

The methylation status of multiple sites within a single CpG island tends to be the same (referred to as co-methylation). Thus a single target site in a CpG island can be enough to get a picture of the whole island's methylation status. The pairing of just two enzymes, HinP1I+AciI, provides >99% coverage of CpG islands with minimal complexity in the reaction mixture and rapid digestion. The same high levels of coverage are provided when using other MSREs which have the same recognition sequences (e.g. HhaI, AspLEI or CfoI in place of HinP1I and/or SsiI in place of AciI), but the preferred combination of AciI and HinP1I offers the advantage that both enzymes can be completely inactivated by heating to 65° C., whereas HhaI and AspLEI are insensitive to heat inactivation. In addition, both AciI and HinP1I use the same optimal buffer for 100% activity, whereas the suppliers' recommended optimal reaction buffers for each of SsiI, AspLEI and CfoI are different, making it more difficult to tailor the digestion conditions for these alternative enzyme combinations without compromising enzyme activity.

HinP1I and AciI can be completely inactivated by heating to 65° C. for 20 minutes, and the improved coverage which comes from adding HpaII or HpaII+HpyCH4IV or HhaI+BstUI+HpaII (as in certain known methods) does not justify the downsides which come from requiring 80° C. for inactivating HpaII. The same is true for the addition of BstUI, which cannot even be heat-inactivated.

HinP1I and AciI were individually used for human cfDNA digestion, followed by qPCR for various loci. To obtain comparable ΔCq values for each enzyme, it was necessary to use more units of HinP1I. It is possible that this is because AciI cuts the human genome more frequently than HinP1I, and a single cut is enough to prevent PCR amplification. Within a mixture of HinP1I+AciI, better results were found when using an excess (in enzyme units) of HinP1I, with an excess between 4-fold and 6-fold being most useful, and a 4.5-fold excess providing the best results (in terms of ΔCq for a variety of different loci vs. a control locus).

The HinP1I+AciI pairing, with an excess of HinP1I, has been used to digest human plasma-derived cfDNA prepared and pooled from about 60 subjects. The purified cfDNA was mixed with the enzymes and incubated at 37° C. for 2 hours, then inactivated at 65° C. for 20 minutes. 2 hours was long enough to achieve complete digestion (except when blood was collected in BCT from Streck, in which case longer periods were typically needed to achieve complete digestion).

A useful digestion mix is prepared by mixing 11 μL rCutSmart™ buffer (10× strength), 4.5 μL HinP1I (10,000 units/mL), 1 μL AciI (10,000 units/mL), and 93.5 μL cfDNA solution (containing the complete cfDNA extracted from a single blood collection tube). Digestion at 37° C. for 2 hours, followed by heating at 65° C. for 20 minutes, provides good results.

The digested cfDNA was used to prepare a sequencing library using NEBNext Ultra DNA Library Prep Kit. The sequencing library was prepared while preserving the information at the ends of the DNA molecules, by adding Illumina platform sequencing adapters using enzymatic ligation. The libraries were subjected to whole-genome NGS using Illumina NovaSeq 6000 sequencing platform with a S4 flow cell. The sequence reads from each sample were mapped against the complete human genome (hg18 genomic build). From over 18×10⁹ sequencing reads, 98.4% were mapped to the reference genome.

Any enzyme with a recognition sequence that is longer than but encompasses the recognition sequence of any of the enzymes listed in Table 1 or Table 2, will inherently have a lower genome-wide CpG coverage/CpG island coverage than the relevant enzyme. The greater the recognition sequence in length, the less likely it will appear in the genome, because a recognition sequence that is more than 4 nucleobases in length is statistically less likely to appear in the genome than a recognition sequence that is 4 nucleobases in length. Thus, combinations of enzymes with a recognition sequence that is longer than but encompasses the recognition sequence of AciI or HinP1I can have a high CpG coverage/CpG island coverage, but this will not be as high as achieved using the preferred combination of AciI and HinP1I. A list of MSRE/MDRE which have a recognition sequence that encompasses the recognition sequence of AciI (CCGC) or HinP1I (GCGC) is provided in Tables 3A and 3B, respectively, below.

TABLE 3A
MSRE/MDRE which have a recognition sequence that
encompasses the recognition sequence of AciI
(CCGC).

| Name | Recognition sequence |
|---|---|
| BsrBI/MbiI | CCGCTC |
| NotI | GCGGCCGC |
| SacII/Cfr42I/SgrBI | CCGCGG |

TABLE 3B
MSRE/MDRE which have a recognition sequence that
encompasses the recognition sequence of HinP1I
(GCGC).

| Name | Recognition sequence |
|---|---|
| AfeI/Aor51HI | AGCGCT |
| AscI | GGCGCGCC |
| BssHII/PauI | GCGCGC |
| HaeII | RGCGCY |
| Eco47III | AGCGCT |
| EheI | GGCGCC |
| FspAI | TGCGCA |
| GlaI | GmCGmC |
| KasI | GCGCGC |
| MteI | G^{m}CG^{m}CNG^{m}CGC |
| NarI | GGCGCC |
| NsbI | TGCGCA |
| PluTI | GGCGCC |
| SfoI | GGCGCC |
| SgsI | GGCGCGCC |

The CpG coverage and CpG island coverage for each of the enzymes listed in Tables 3A and Table 3B is lower than for AciI and HinP1I, respectively.

It will be understood that the inventors' work has been described above by way of example only and modifications may be made while remaining within the scope and spirit of the invention.

### REFERENCES

WO 2005/090607
WO 2011/109529
WO 2014/078913
WO 2015/169947
WO 2020/188561
WO 2022/073012
WO 2022/107145
U.S. Pat. No. 10,801,060
US 2020/0283840
Aleman et al. (2008) Br J Cancer; 98 (2), 466-473
Alhonen-Hongisto et al. (1987) Biochem J; 242, 205-210
Bait et al. (2020) Genome; 64 (5): 533-546
Bartlett et al. (1991) Somatic Cell & Molecular Genetics; 17 (1): 35-47
Beikircher et al. (2018) Chapter 21 in DNA Methylation Protocols (ed. Jörg Tost), Methods in Molecular Biology, vol. 1708:407-424
Ellinger et al. (2009) J Urol 182:324-29
Ghosh & Sen-Mandi (2018) Tropical Plant Research; 5 (1): 1-7
Gong et al. (2002) Cell; 11 (6); 803-814
Khulan et al. (2006) Genome Res 16:1046-55
Larsson et al. (2012) Tree Genetics & Genomes; 9:601-612
List et al. (1994) J. Biological Chemistry; 269 (16): 11902-11911
Nalabothula et al. (2015) PLoS ONE 10 (8): e0135410
Schmidt et al. (2017) Clinica Chimica Acta 469:94-8
Van Roon et al. (2013) Clinical Epigenetics; 5 (2): 1-10
van Zogchel et al. (2021) JCO Precision Oncology 5:1738-1748
Wielscher et al. (2015) EbioMedicine 2:929-36
Yan et al. (2009) Chapter 8 in DNA Methylation: Methods and Protocols (ed. Jörg Tost), Methods
Molecular Biology; 507:89-106
Zhao et al. (2010) Prenat Diagn 30:778-82

1. A method for digesting cfDNA using a combination of restriction enzymes consisting of HinP1I and AciI, wherein the method comprises steps of: (i) digesting the cfDNA with the restriction enzymes; and (ii) inactivating the restriction enzymes by heating for longer than 15 minutes, wherein the restriction enzymes are completely inactivated by the heating.

2. A method for digesting cfDNA using a combination of restriction enzymes consisting of HinP1I and AciI, wherein the method comprises steps of: (i) providing a blood sample contained within a collection tube that includes an anticoagulant and an agent to inhibit genomic DNA from white blood cells in the sample being released into the plasma component of the blood sample; (ii) preparing plasma from the blood sample; and (iii) digesting the plasma cfDNA with the restriction enzymes for at least 2 hours.

3. A method for analysing cfDNA, wherein the method comprises steps of: (i) digesting the cfDNA using a combination of restriction enzymes consisting of HinP1I and AciI, to provide digested cfDNA; and (ii) sequencing of the digested cfDNA.

4. A method for digesting cfDNA using a combination of restriction enzymes comprising HinP1I and AciI, wherein the method comprises steps of: (i) digesting the cfDNA with the restriction enzymes for 11 hours or less; and (ii) inactivating the restriction enzymes by heating, wherein the restriction enzymes are completely inactivated by the heating.

5. A method for analysing cfDNA, wherein the method comprises steps of: (i) digesting the cfDNA for 11 hours or less using a combination of restriction enzymes comprising HinPII and AciI, to provide digested cfDNA; (ii) completely inactivating the restriction enzymes; and (iii) performing real time PCR on the digested cfDNA.

6. The method of claim 5, wherein the combination of restriction enzymes consists of HinPII and AciI.

7. A composition comprising HinP1I and AciI as the only two restriction enzymes in the composition.

8. A composition comprising HinP1I and AciI, wherein the ratio of activity of HinP1I to AciI is at least 1.2:1.

9. A composition comprising a plurality of restriction enzymes, wherein the plurality consists of MSRE and/or MDRE, wherein (i) at least two different restriction enzymes in the plurality have different recognition sequences, and (ii) the restriction enzymes can be completely inactivated by heating to 65° C.

10. The composition of claim 9, comprising a plurality of MSREs wherein (i) at least two different MSREs in the plurality have different recognition sequences, and (ii) the plurality of MSREs can be completely inactivated by heating to 65° C.

11. The composition of claim 7 wherein the composition further comprises at least one salt and/or at least one buffer; for instance, including (i) 50 mM potassium acetate, 20 mM Tris-acetate, 10 mM magnesium acetate, 100 μg/mL recombinant albumin, pH 7.9 or (ii) 50 mM Tris-HCl, 10 mM MgCl2, 100 mM NaCl, 100 μg/mL recombinant albumin, pH 7.9.

12. The composition of claim 7, wherein the composition further comprises cfDNA.

13. The composition of claim 7, including PCR reagents and/or sequencing reagents.

14. The composition of claim 7, wherein: (a) the ratio of HinP1I to AciI is at least 2:1; (b) the composition further comprises Mg++ ions; and/or (c) the pH of the composition is above 7.

15. A method for analysing cfDNA, comprising digesting the cfDNA by the method of claim 1, followed by (a) performing real-time PCR on the digested cfDNA or (b) sequencing the digested cfDNA.

16. A method for assessing methylation status of one or more CpG sites in cfDNA, comprising digesting the cfDNA by the method of claim 1, followed by quantifying a degree of digestion at one or more of the one or more CpG sites.

17. A method for diagnosing the presence of absence of a cancer in a subject, comprising assessing methylation status of one or more CpG sites in cfDNA from the subject by the method of claim 16, wherein hypermethylation and/or hypomethylation of the one or more CpG sites is associated with the cancer.

18. A method for treating or managing a cancer in a subject, comprising diagnosing the presence of cancer in the subject by the method of claim 17, and administering a suitable anti-cancer treatment to the subject.

19. The method of claim 1, wherein the restriction enzymes are inactivated after cfDNA digestion by heating the composition to 65° C. for at least 20 minutes, wherein the restriction enzymes are completely inactivated by the heating.

20. The method of claim 1, wherein: (a) the activity ratio of HinP1I to AciI is at least 2:1; (b) the restriction enzymes are provided with a source of Mg++ ions during digestion; (c) digestion occurs at a pH above 7; (d) the cfDNA is human cfDNA; and/or (e) the amount of cfDNA subjected to digestion is between 10 400 ng.

21. The method of claim 1, wherein cfDNA is human plasma cfDNA.

22. The method of claim 1, wherein HinP1I is present at an excess (measured in terms of enzymatic units) to AciI of between 2:1 and 5:1.

23. The method of claim 1, wherein HinP1I is replaced with a MSRE which recognises the same recognition sequence as HinP1I, and/or wherein AciI is replaced with a MSRE which recognises the same recognition sequence as AciI.

24. A composition comprising (i) HinP1I, or a MSRE that recognises the same recognition sequence as HinP1I, and (ii) AciI, or a MSRE that recognises the same recognition sequence as AciI, as the only two restriction enzymes in the composition.

25. The composition of claim 24, wherein (i) the MSRE that recognises the same recognition sequence as HinP1I is HhaI, AspLEI or CfoI nd/or (ii) the MSRE that recognises the same recognition sequence as AciI is SsiI.

26. A composition comprising (i) any one of HhaI, AspLEI and CfoI and (ii) SsiI as the only two restriction enzymes in the composition.
